(19) United States
(12) Patent Application Publication
(10) Pub. No.: US 2011/0105946 A1
(43) Pub. Date: May 5, 2011

(54) BIOPSY SYSTEM WITH INFRARED COMMUNICATIONS

(76) Inventors: Peter L. Sorensen, Vipperod (DK); Claus Reuber, Roskilde (DK)

(21) Appl. No.: 12/610,278

(22) Filed: Oct. 31, 2009

(51) Int. Cl. A61B 10/02

(52) U.S. Cl. 600/567

(57) ABSTRACT

A biopsy system includes a host and a biopsy driver assembly. The host is configured to execute program instructions associated with an application. The host has a first IrDA interface. The biopsy driver assembly has a controller for executing program instructions and a user interface providing user input to the controller. The biopsy driver assembly has a second IrDA interface. The second IrDA interface is default disabled. The controller of the biopsy driver assembly has sole control in enabling the second IrDA interface to in turn enable an infrared communications link between the first IrDA interface of the host and the second IrDA interface of the biopsy driver assembly.

### CROSS-REFERENCE TO RELATED APPLICATIONS

None.

### MICROFICHE APPENDIX

None.

### GOVERNMENT RIGHTS IN PATENT

None.

### BACKGROUND OF THE INVENTION

1. Field of the Invention

The present invention relates to a biopsy apparatus, and, more particularly, to a biopsy system with infrared communications.

2. Description of the Related Art

A biopsy may be performed on a patient to help in determining whether the cells in a biopsied region are cancerous. One type of vacuum assisted biopsy apparatus includes a hand-held biopsy driver assembly having a vacuum source, and a disposable biopsy probe assembly configured for releasable attachment to the driver assembly. One biopsy technique used to evaluate breast tissue, for example, involves inserting a biopsy probe into the breast tissue region of interest to capture one or more tissue samples from the region.

The biopsy probe typically includes a biopsy cannula, e.g., a needle, having a cylindrical side wall defining a lumen, and having a side sample notch located near the distal end that extends though the side wall to the lumen. A cutting cannula is positioned coaxial with the biopsy cannula to selectively open and close the sample notch. Vacuum is applied to the lumen, and in turn to the sample notch, for receiving the tissue to be sampled when the sample notch is opened, after which the sample notch is closed by the cutting cannula to sever the tissue, and the severed tissue is transported by vacuum out of the lumen and collected.

In some circumstances, it may be desirable to communicate with the biopsy driver assembly using a remote device, such as a host (i.e., a personal computer). However, wired links, such as a wired USB connection, leads to a mechanical solution with openings to a connector on the device. As such, there is a safety risk of inducing electrical signals on the USB connector terminals which could harm biopsy driver assembly and/or bring the biopsy driver assembly in an undefined state. Another disadvantage of a wired connection is that moisture could enter the device through the connector. Also, a short range radio frequency (RF) wireless standard is a complex solution, and has disadvantages with respect to electromagnetic compatibility (EMC), electromagnetic interference (EMI) and the size of solutions.

### SUMMARY OF THE INVENTION

The present invention provides for the selective establishing of an infrared communications link between a host, such as a personal computer, and a biopsy driver assembly.

As used herein, the terms “first” and “second” preceding an element name, e.g., first IrDA interface and second IrDA interface, etc., are for identification purposes to distinguish between different elements having similar characteristic, and are not intended to necessarily imply order, unless otherwise specified, nor are the terms “first”, “second”, etc., intended to preclude the inclusion of additional similar elements.

The invention, in one form thereof, is directed to a biopsy system. The biopsy system includes a host and a biopsy driver assembly. The host is configured to execute program instructions associated with an application. The host has a first IrDA interface. The biopsy driver assembly has a controller for executing program instructions and a user interface providing user input to the controller. The biopsy driver assembly has a second IrDA interface. The second IrDA interface is default disabled. The controller of the biopsy driver assembly has sole control in enabling the second IrDA interface to in turn enable an infrared communications link between the first IrDA interface of the host and the second IrDA interface of the biopsy driver assembly.

The invention, in another form thereof, is directed to a biopsy system. The biopsy system includes a host and a biopsy driver assembly. The host is configured to execute program instructions associated with an application, the host having a host memory. A biopsy driver assembly has a controller, firmware, a driver memory, and an event log established in the driver memory. The firmware has program instructions which when executed by the controller update the event log to record events related to usage of the biopsy driver assembly. An infrared communications link facilitates communication between the host and the biopsy driver assembly. The host executes program instructions from the application to retrieve the event log from the biopsy driver assembly over the infrared communications link.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of a biopsy apparatus, configured in accordance with an embodiment of the present invention, with a disposable biopsy probe assembly mounted to a biopsy driver assembly;

FIG. 2 is a perspective view of the biopsy apparatus of FIG. 1, with the disposable biopsy probe assembly detached from the biopsy driver assembly;

FIG. 3 is a block diagram showing various components of the biopsy driver assembly and biopsy probe assembly of FIG. 1, and schematically illustrating a mechanical connection between components of the biopsy driver assembly and the biopsy probe assembly to form the biopsy apparatus of FIG. 1;

FIG. 4 is a block diagram illustrating an infrared communication link established between a host, such as a personal computer, and the biopsy driver assembly of FIG. 2; and

FIG. 5 is a block diagram showing the details of the IrDA interface of the biopsy driver assembly of FIG. 4 in communication with a microcontroller unit of the biopsy driver assembly of FIG. 4.

Corresponding reference characters indicate corresponding parts throughout the several views. The exemplifications set out herein illustrate an embodiment of the invention, and such exemplifications are not to be construed as limiting the scope of the invention in any manner.

### DETAILED DESCRIPTION OF THE INVENTION

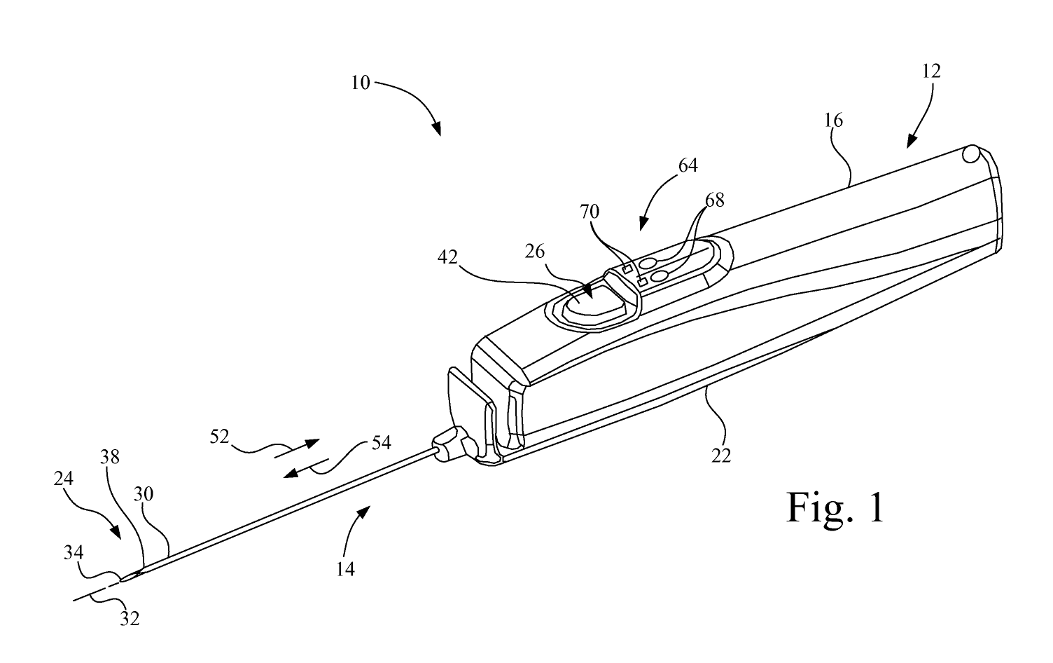

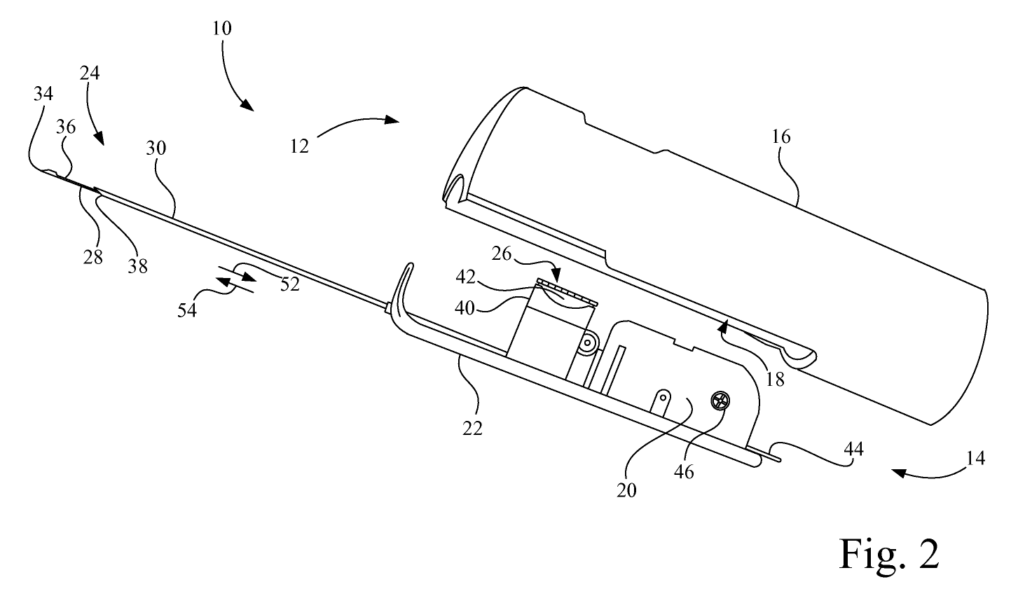

Referring now to the drawings, and more particularly to FIGS. 1 and 2, there is shown a biopsy apparatus 10 which generally includes a non-invasive, e.g., non-disposable, biopsy driver assembly 12 and a disposable biopsy probe assembly 14. As used herein, the term “non-disposable” is used to refer to a device that is intended for use on multiple patients during the lifetime of the device, and the term “disposable” is used to refer to a device that is intended to be disposed of after use on a single patient. Biopsy probe assembly 14 is configured for releasable attachment to biopsy driver assembly 12. As used herein, the term “releasable attachment” means a configuration that facilitates an intended temporary connection followed by selective detachment involving a manipulation of disposable biopsy probe assembly 14 relative to biopsy driver assembly 12, without the need for tools.

Biopsy driver assembly 12 includes a housing 16 configured, and ergonomically designed, to be grasped by a user. Housing 16 defines an elongate cavity 18 which is configured for receiving a corresponding housing 20 of biopsy probe assembly 14 when biopsy driver assembly 12 is mounted to biopsy probe assembly 14.

Biopsy probe assembly 14 includes housing 20, a cover 22, a biopsy probe 24, and a tissue sample retrieval mechanism 26. Biopsy probe 24 is mounted to housing 20, and housing 20 is mounted to cover 22. Cover 22 serves as a slidable cover to close elongate cavity 18 in housing 16 of biopsy driver assembly 12 to protect the internal structure of biopsy driver assembly 12 when biopsy probe assembly 14 is mounted to biopsy driver assembly 12.

Biopsy probe 24 includes a sample basket 28 and a cutter cannula 30. Each of sample basket 28 and cutter cannula 30 is configured to be individually movable along a longitudinal axis 32. Sample basket 28 of biopsy probe assembly 14 has a sharpened tip 34 to aid in puncturing tissue and has a sample notch 36 in the form of a recessed region for receiving a biopsy tissue sample. Cutter cannula 30 of biopsy probe assembly 14 has a sharpened distal end 38 to aid in severing tissue received in sample basket 28.

Tissue sample retrieval mechanism 26 includes a sample tank receptacle 40 and a sample collection tank 42. Sample tank receptacle 40 may be formed integral with and/or as a part of housing 20. Sample collection tank 42 is slidably received in sample tank receptacle 40. Sample collection tank 42 is configured as a receptacle having an open interior with a lower port (not shown) leading to the open interior. A tissue sample is received by the lower port and is delivered into the open interior by advancement of the tissue sample relative to sample collection tank 42.

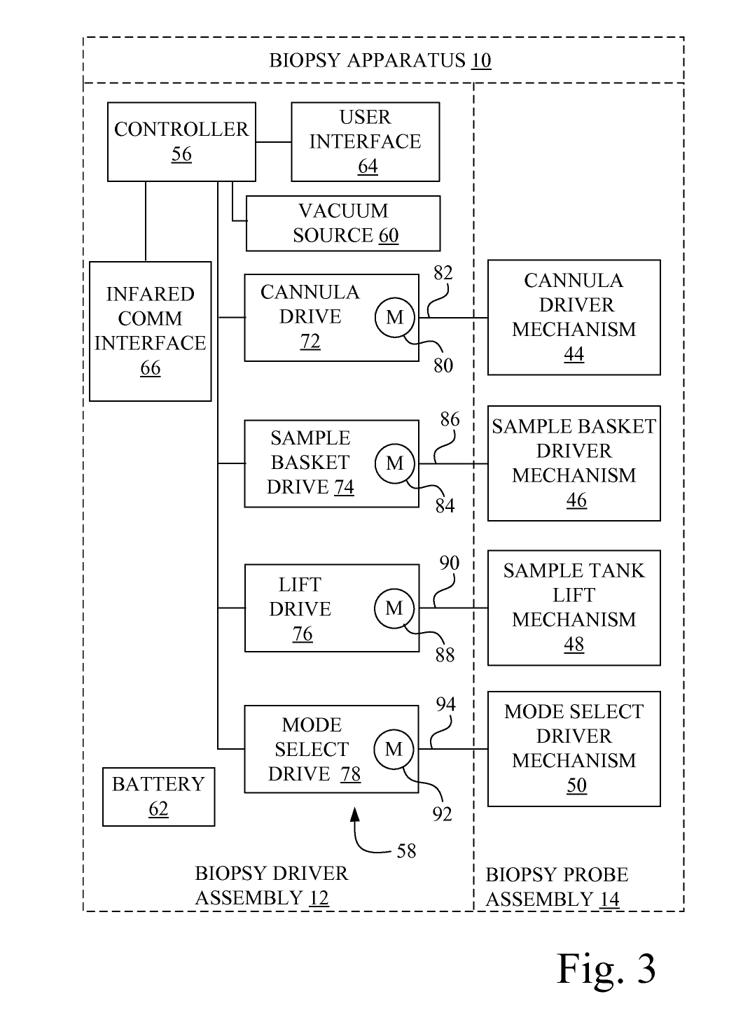

Referring also to FIG. 3, biopsy probe assembly 14 further includes a cannula driver mechanism 44, a sample basket driver mechanism 46, a sample tank lift mechanism 48, and a mode select driver mechanism 50.

A cannula driver mechanism 44 is drivably coupled to cutter cannula 30 to facilitate movement of cutter cannula 30 along longitudinal axis 32 in either of direction 52 or direction 54. Cannula driver mechanism 44 may be in the form of an elongate slide that is slidably coupled to housing 20. The sliding coupling of cannula driver mechanism 44 to housing 20 may be achieved by placing cannula driver mechanism 44 in a longitudinal slide channel (not shown) formed in housing 20.

Sample basket driver mechanism 46 is drivably coupled to sample basket 28 to facilitate movement of sample basket 28 along longitudinal axis 32 in either of directions 52 or 54. Sample basket driver mechanism 46 is contained, at least in part, in housing 20. Sample basket driver mechanism 46 includes a gear train (not shown) that converts rotary motion to linear motion, such as for example, a flexible toothed rack that is connected to sample basket 28, and a gear unit having a gear that drivably engages the toothed rack.

Sample tank lift mechanism 48 is configured to lift sample collection tank 42 away from longitudinal axis 32. Such lifting may be effected, for example, by using a movable ramp that engages a portion of sample collection tank 42 as the ramp moves in direction 52 while collection tank is retained horizontally stationary in directions 52 and 54 by sample tank receptacle 40. Likewise, movement of the ramp along longitudinal axis 32 in direction 54 opposite to direction 52 will lower sample collection tank 42 toward longitudinal axis 32.

Mode select driver mechanism 50 is configured to select, i.e., switch, between a tissue harvesting mode and a piercing shot mode. Mode select driver mechanism 50 is configured such that, in the tissue harvesting mode, cannula driver mechanism 44 is able to move cutter cannula 30 independent of sample basket 28, such that, for example, cannula driver mechanism 44 attached to cutter cannula 30 may be advanced relative to sample basket 28 to sever tissue present in sample basket 28. Likewise, sample basket driver mechanism 46 is able to move sample basket 28 independent from cutter cannula 30, such that, for example, sample basket 28 may be retracted within cutter cannula 30 to deliver the severed tissue sample to sample collection tank 42.

Mode select driver mechanism 50 further is configured such that, in the piercing shot mode, cutter cannula 30 and sample basket 28 move in unison, e.g., locked together, for linear travel along longitudinal axis 32. For example, mode select driver mechanism 50 may include a slide mechanism (not shown), for selectively coupling cannula driver mechanism 44 to sample basket driver mechanism 46.

Referring also to FIG. 3, biopsy driver assembly 12 contains within housing 16 a controller 56, a plurality of electromechanical drives 58, a motorized vacuum source 60, and a rechargeable battery 62. Mounted within and exposed through housing 16 is a user interface 64 and an infrared communications interface 66. Battery 62 provides electrical power to all electrically powered components in biopsy driver assembly 12, and thus for simplicity in the drawings, such electrical couplings are not shown. For example, battery 62 is electrically coupled to controller 56, the plurality of electromechanical drives 58, motorized vacuum source 60, user interface 64, and infrared communications interface 66.

User interface 64 is communicatively coupled to controller 56. The user interface 64 includes control buttons 68 and visual indicators 70. Control buttons 68 provide user control over various functions supported by biopsy driver assembly 12, including enabling infrared communications interface 66 for external communications. Visual indicators 70 provide visual feedback of the status of one or more conditions and/or positions of components of biopsy apparatus 10.

Controller 56 further is communicatively coupled to each of the plurality of electromechanical drives 58, motorized vacuum source 60 and to infrared communications interface 66. Controller 56 may include, for example, a microcontroller and associated memory for executing program instructions to perform functions associated with the retrieval of biopsy tissue samples, such as by controlling one or more the plurality of electromechanical drives 58 and motorized vacuum source 60, and may execute program instructions to monitor one or more conditions and/or positions of components of biopsy apparatus 10. Further, controller 56 may execute program instructions for establishing communications with an external device via infrared communications interface 66.

In the present embodiment, plurality of electromechanical drives 58 includes a cannula drive 72, a sample basket drive 74, a lift drive 76 and a mode select drive 78, each being respectively coupled to battery 62, and each of drives 72, 74, 76 and 78 being respectively electrically and controllably coupled to user interface 64 via controller 56.

Cannula drive 72 may include an electrical motor 80 coupled to a motion transfer unit 82 (shown schematically by a line) by one or more of a gear, gear train, belt/pulley arrangement, etc. Electrical motor 80 may be, for example, a stepper motor, a direct current (DC) motor, etc. Motion transfer unit 82 of cannula drive 72 is configured for coupling to cannula driver mechanism 44 of biopsy probe assembly 14. Motion transfer unit 82 may be configured, for example, with a rotational-to-linear motion converter, such as a worm gear arrangement, rack and pinion arrangement, etc., or a solenoid-slide arrangement, etc., to compress a spring in cannula drive 72. The spring in cannula drive 72 stores energy when the spring is compressed, and releases the stored energy when decompressed.

In the tissue harvesting mode, for example, cannula drive 72 releases the stored energy to propel, i.e., move in a rapid abrupt manner, cannula driver mechanism 44 to move cutter cannula 30 independent of the linearly stationary sample basket 28 to sever tissue in sample basket 28. In the piercing shot mode, cannula drive 72 releases the stored energy to propel (fire) cutter cannula 30 and sample basket 28 in unison to aid in inserting biopsy probe 24 into fibrous tissue.

Sample basket drive 74 may include an electrical motor 84 coupled to a motion transfer unit 86 (shown schematically by a line) by one or more of a gear, gear train, belt/pulley arrangement, etc. Electrical motor 84 may be, for example, a stepper motor, a direct current (DC) motor, etc. Motion transfer unit 86 of sample basket drive 74 may be configured to transmit rotary motion, such as one or more of a gear, gear train, belt/pulley arrangement, etc., to drive sample basket driver mechanism 46.

Motion transfer unit 86 is configured for coupling to sample basket driver mechanism 46 of biopsy probe assembly 14 to move sample basket 28 along longitudinal axis 32 in either of directions 52 or 54. For example, after a tissue sample is severed by cutter cannula 30, motion transfer unit 86 moves sample basket 28 to the location of sample collection tank 42 of tissue sample retrieval mechanism 26 to transfer the tissue sample to sample collection tank 42.

Lift drive 76 may include an electrical motor 88 coupled to a motion transfer unit 90 (shown schematically by a line) by one or more of a gear, gear train, belt/pulley arrangement, etc. Electrical motor 88 may be, for example, a stepper motor, a direct current (DC) motor, etc. Motion transfer unit 90 of lift drive 76 may include one or more of a gear, gear train, belt/pulley arrangement, etc.

Motion transfer unit 90 is configured for coupling to sample tank lift mechanism 48 of biopsy probe assembly 14 to effect a linear translation of the ramp of sample tank lift mechanism 48 used in the lifting and lowering of sample collection tank 42. For example, when motion transfer unit 86 moves sample basket 28 to the location of sample collection tank 42, motion transfer unit 90 operates sample tank lift mechanism 48 to lower sample collection tank 42 and scoop the tissue sample out of sample basket 28.

Mode select drive 78 may include an electrical motor 92 coupled to a motion transfer unit 94 (shown schematically by a line) by one or more of a gear, gear train, belt/pulley arrangement, etc. Electrical motor 92 may be, for example, a stepper motor, a direct current (DC) motor, etc. Motion transfer unit 94 may be configured as a motor driven linear motion converter, such as for example a worm gear arrangement, rack and pinion arrangement, etc., or alternatively, may provide linear motion be a solenoid-slide arrangement.

Motion transfer unit 94 of mode select drive 78 is configured for coupling to mode select driver mechanism 50 of biopsy probe assembly 14 to facilitate a linear movement of the slide mechanism in mode select driver mechanism 50 to select between the tissue harvesting mode and the piercing shot mode. For example, movement of the slide mechanism in mode select driver mechanism 50 in direction 52 may select the piercing shot mode, whereas movement of the slide mechanism in mode select driver mechanism 50 in direction 54 may select the tissue harvesting mode.

In a biopsy procedure, under the control of controller 56, mode select drive 78 selects the piercing shot mode via mode select driver mechanism 50, and cannula drive 72 operates cannula driver mechanism 44 to fire sample basket 28 and cutter cannula 30 in unison into the tissue to be biopsied. The piercing shot mode is optional, as determined by the physician conducting the biopsy procedure.

Then, mode select drive 78 selects the tissue harvesting mode via mode select driver mechanism 50. After the biopsy probe 24 is positioned at the proper depth and orientation with respect to the specific tissue area to be biopsied, cutter cannula 30 is linearly driven by cannula drive 72 via cannula driver mechanism 44 to traverse over sample notch 36 of sample basket 28 along longitudinal axis 32 in direction 52 to expose sample notch 36. Vacuum source 60, having been coupled to a vacuum conduit in fluid communication with sample notch 36, is activated to draw tissue into sample notch 36. To harvest the tissue sample, cutter cannula 30 is linearly driven by cannula drive 72 via cannula driver mechanism 44 to traverse over sample notch 36 of sample basket 28 along longitudinal axis 32 in direction 54 to sever the tissue prolapsed into sample notch 36. Thereafter, sample basket 28 is retracted by sample basket drive 74 via sample basket driver mechanism 46 along longitudinal axis 32 in direction 52 to the location of sample collection tank 42, which in turn is lowered by operation of lift drive 76 via sample tank lift mechanism 48 to scoop the tissue sample out of sample notch 36 as sample basket 28 continues to move in direction 52. If multiple samples are desired from the patient, then biopsy apparatus 10 is reset, and the procedure outlined above may be repeated.

Although biopsy probe assembly 14 may be used to collect multiple tissue samples from a single patient, biopsy probe assembly 14 is disposable and is not intended for use with multiple patients. In contrast, biopsy driver assembly 12 is intended to be use with multiple patients, and may be used with multiple types of biopsy probe assemblies.

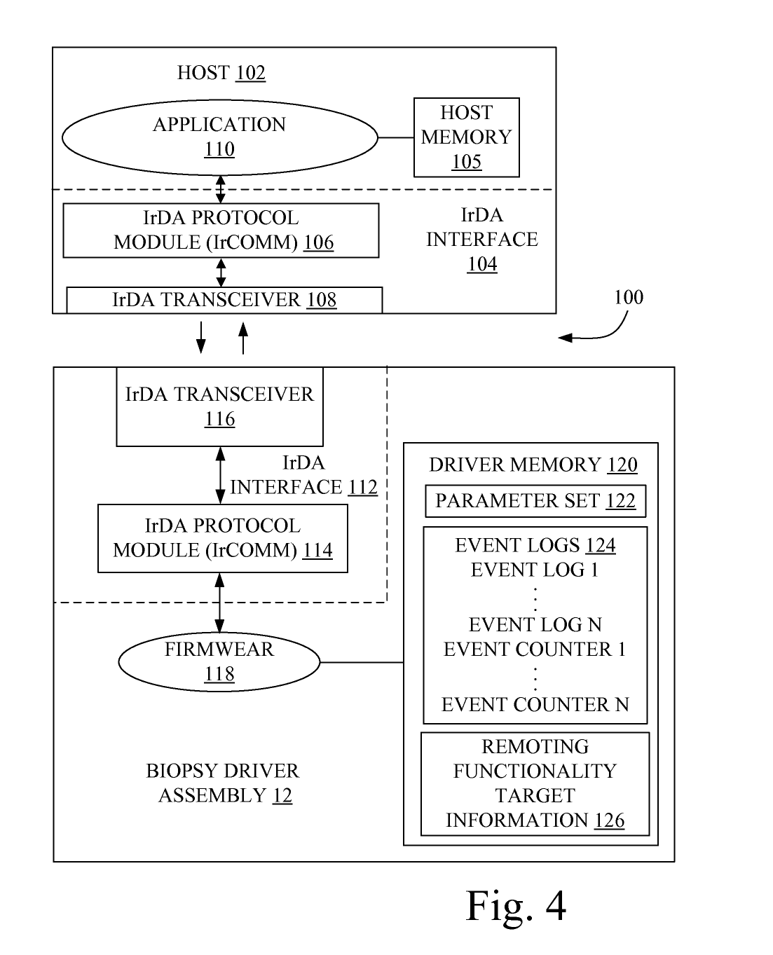

In accordance with an aspect of the present invention, with reference to FIG. 4, an infrared communications link 100 may be established between a host 102 and biopsy driver assembly 12 to facilitate bidirectional communications between biopsy driver assembly 12 and host 102. Infrared communications link 100 is based, for example, on the Infrared Data Association (IrDA) standard. Information that may be communicated over infrared communications link 100 includes, for example, event logs associated with a patterns of use of biopsy driver assembly 12, device parameters to be downloaded from host 102 to biopsy driver assembly 12 during production assembly, and remoting commands to facilitate remote control of device functions of biopsy driver assembly 12 during production and/or while in service for testing via host 102.

In general, it was found that infrared communications link 100 has an advantage for use with biopsy driver assembly 12 over that of wired links, such as a wired USB connection, since wired USB leads to a mechanical solution with openings to a connector on the device. As such, infrared communications link 100 avoids a safety risk of inducing any electrical signals on wired USB connector terminals which could harm biopsy driver assembly 12 and/or bring biopsy driver assembly 12 in an undefined state. In addition, infrared communications link 100 avoids the disadvantage of a wired connection in which moisture could enter the device through the connector.

Also, it was found that infrared communications link 100 has an advantage for use with biopsy driver assembly 12 over that of short range radio frequency (RF) wireless, since an RF wireless standard is a complex solution, and has disadvantages with respect to electromagnetic compatibility (EMC), electromagnetic interference (EMI) and the size of solutions.

Host 102 may be, for example, a personal computer, including host memory 105, such as random access memory (RAM), read only memory (ROM), and/or nonvolatile RAM (NVRAM), an input device, such as a keyboard, and a display monitor. Host 102 further includes a microprocessor and typically at least one mass data storage device, such as a hard drive, a CD-ROM and/or a DVD unit, and input/output (I/O) interfaces. In the present embodiment, host 102 includes an I/O interface in the form of an IrDA interface 104 as the host-side portion of infrared communication link 100, which is schematically illustrated has having a standardized infrared communication protocol such as an IrDA protocol module (IrCOMM) 106 and an IrDA transceiver 108. IrDA interface 104 may be implemented, for example, as a commercially available IrDA universal serial bus (USB) dongle.

An application 110, i.e., a software program, may be placed in host memory 105 for execution by host 102. Application 110 includes program instructions to be executed by host 102 to facilitate bidirectional communication over infrared communications link 100 via IrDA interface 104. Application 110 includes program instructions to provide data safety with checksum and data echo for verification of information retrieved from, or transferred to, biopsy driver assembly 12.

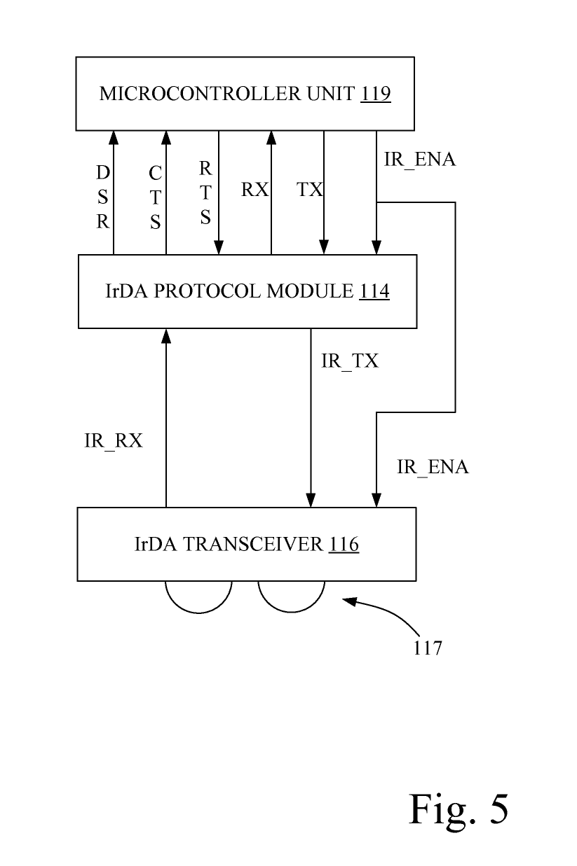

Biopsy driver assembly 12 includes an input/output (I/O) interface in the form of an IrDA interface 112 suitable for use as infrared communications interface 66 (see FIG. 3) as the driver-side portion of infrared communication link 100, which is schematically illustrated has having a standardized infrared communication protocol, i.e., IrDA protocol module (IrCOMM) 114 and an IrDA transceiver 116 (see also FIG. 5).

IrDA protocol module 114 may be, for example, a MCP2155 IrDA Protocol Stack Handler available from Microchip Technology Incorporated. IrDA protocol module 114 establishes and controls the low level IrDA communication between biopsy driver assembly 12 and host 102.

IrDA transceiver 116 may be a TFDU4300-TR1 available from Vishay Semiconductors. IrDA transceiver 116 serves as the interface between electrical signals and infrared light source 117.

Biopsy driver assembly 12 includes firmware 118 (see FIG. 4) having program instructions which when executed by a microcontroller 119 (see FIG. 5) facilitates bidirectional communication over infrared communications link 100 via IrDA interface 112, and further executes to read/write data from/to a driver memory 120. Firmware 118 may be resident in NVRAM and formed as part of a microcontroller 119, which in turn may be formed as a part of the overall controller 56 (see also FIG. 3). Microcontroller 119 may be, for example, an ATmega64 available from Atmel Corporation.

As shown in FIG. 5, microcontroller 119 is coupled to IrDA protocol module 114 (IrCOMM) via communication lines DSR, CTS, RTS, RX, TX, and IR_ENA. Microcontroller 119 is also communicatively coupled to IrDA transceiver 116 via communication line IR_ENA. IrDA protocol module 114 is in turn communicatively coupled to IrDA transceiver 116 via communication lines IR_RX and IR_TX. IrDA protocol module 114 functions as a converter between the microcontroller 119 signals and the IrDA signals. Microcontroller 119 controls all functionalities related to IrDA communication. All serial communication with the IrDA system is done through an implemented universal synchronous asynchronous receiver transmitter (USART) port, and data flow control signals are controlled, for example, by general purpose input/output (GPIO) pins and one GPIO pin controls enabling and disabling of the IrDA circuit formed by IrDA protocol module 114 and IrDA transceiver 116. The IrDA circuit (IrDA interface 112) is as default disabled via IR_ENA and is only enabled when the external IrDA communication mode is entered by technicians via user interface 64.

Referring again to FIG. 4, driver memory 120 may be partitioned to include, for example, a section for storing a parameter set 122, a section for storing event logs 124 (event log 1 through event log N; and event counter 1 through event counter N), and a section for storing remoting functionality target information 126. As used herein, the term “remoting” refers to the remote operation of biopsy driver assembly 12 by host 102.

The parameter set 122 may include, for example, a serial number of biopsy driver assembly 12, a firmware version identification number of biopsy driver assembly 12, a real time clock setting of biopsy driver assembly 12, and motor positions of a plurality of motors, e.g., electrical motors 80, 84, 88 and 92, of biopsy driver assembly 12.

The event logs 124 store data associated with a date and time of an occurrence of a respective biopsy event. A biopsy event may include, for example, an event associated with a tissue sample harvesting operation and/or an event associated with a piercing shot operation. More specifically, the biopsy event may be the actuation of one or more of cannula drive 72, sample basket drive 74, lift drive 76, and mode select drive 78. The event logs 124 also store event counters (event counter 1 through event counter N) associated with a respective biopsy event. The event counters may also be referred to as lifetime counters, since each event counter maintains a lifetime count of the monitored component. Firmware 118 has program instructions which when executed by microcontroller 119 update the respective event log 1-N to record events related to usage of biopsy driver assembly 12.

The remoting functionality target information 126 identifies target devices within biopsy driver assembly 12 that may be accessed by host 102 to enable automatic testing of biopsy driver assembly 12, such as in the production facilities and to facilitate ease the debugging and testing biopsy driver assembly 12 while in the service.

Biopsy driver assembly 12, through firmware 118 and microcontroller 119, has sole control in enabling infrared communications link 100 by controlling the enable state of IrDA interface 112 via IR_ENA. For example, IrDA protocol module 114 (IrCOMM) is as default disabled and is only enabled by a specific command entered at user interface 64 of biopsy driver assembly 12. Also, biopsy driver assembly 12 may be configured such that infrared communication between host 102 and biopsy driver assembly 12 cannot occur while a biopsy probe assembly 14 is installed on biopsy driver assembly 12. As a further safeguard, application 110 executing on host 102 facilitates password protected access to biopsy driver assembly 12.

Once IrDA interface 112 of biopsy driver assembly 12 is enabled, communication over infrared communications link 100 between host 102 and biopsy driver assembly 12 can commence.

Application 110 of host 102 provides a plurality of pull down menus in a known fashion to aid the user in accessing information from biopsy driver assembly 12 during information retrieval and parameter setting operations, and/or to aid in controlling functions of biopsy driver assembly 12 during remoting operations.

Host 102 executes program instructions from application 110 to selectively read (i.e., retrieve) one or more parameters in parameter set 122 over infrared communications link 100. For example, some parameters in parameter set 122 may be associated with a respective motor of the plurality of motors 80, 84, 88 and 92 of biopsy driver assembly 12. The parameters may be, for example, motor position, e.g., stepper motor counts, used to position the respective drives 72, 74, 76, 78 of the plurality of electromechanical drives 58 of biopsy driver assembly 12, which in turn will drive the respective driver mechanisms 44, 46, 48, and 50, respectively, of biopsy probe assembly 14 (see FIG. 3). Other parameters that host 102 may retrieve from parameter set 122 include the serial number of biopsy driver assembly 12, a firmware version identification number of biopsy driver assembly 12, a real time clock setting of biopsy driver assembly 12, etc.

Similarly, host 102 may execute program instructions from application 110 to selectively modify one or more parameters in parameter set 122 over infrared communications link 100. For example, host 102 may execute program instructions from application 110 to selectively modify one or more parameters associated with a respective motor of the plurality of motors 80, 84, 88 and 92 of biopsy driver assembly 12. Modification of motor parameters may be desirable, for example, to accommodate different valid types of biopsy probe assembly 14.

Host 102 may also execute program instructions from application 110 to retrieve one or more of event logs 124 from biopsy driver assembly 12 over infrared communications link 100. Host 102 may further execute program instructions from application 110 to analyze the plurality of event logs 124 to determine an overall pattern of usage of biopsy driver assembly 12.

In addition, host 102 executes program instructions from application 110 to invoke the remoting operation, so as to selectively control a plurality of functions of biopsy driver assembly 12 from host 102. The remoting operation may occur, for example, to perform tests on biopsy driver assembly 12 during production assembly of biopsy driver assembly 12, or to service biopsy driver assembly 12 after delivery to a customer. The plurality of functions may include, for example, the testing each of the plurality of motors 80, 84, 88, and 92 and the associated plurality of electromechanical drives 58, including drives 72, 74, 76, 78, respectively, in biopsy driver assembly 12. The testing may include at least one of conducting driver operation sequences of the plurality of electromechanical drives 58, measuring motor currents of the plurality of motors 80, 84, 88, and 92, and performing automatic motor adjustment and calibration of one or more of the plurality of motors 80, 84, 88, and 92 by changing motor position parameters in parameter set 122.

To reduce the quantity of data transferred over infrared communications link 100, host 102 has stored in host memory 105 a respective descriptive file for each of a plurality of different types of biopsy driver assembly 12, with each type of biopsy driver assembly 12 being identified by a unique driver identification number. More particularly, the descriptive file includes a listing of: a number of parameters, parameter data types, read-only restrictions, and a description of parameters in parameter set 122 that is associated with a specific biopsy driver type of the plurality of different types of the biopsy driver assembly 12; a number of event counters 1-N, a data type for each respective event counter 1-N, and a description of each respective event associated with the specific biopsy driver type; a number of rows in each event log 1-N of event logs 124, a data type for each respective event log 1-N, and an event index table having descriptions of each respective event log 1-N associated with the specific biopsy driver type; a password to be used for logging onto each type of biopsy driver assembly 12; a proprietary binary format used to read and change data associated with the specific biopsy driver type; and a checksum to check for data consistency before using the descriptive file associated with the specific biopsy driver type of biopsy driver assembly 12.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A biopsy system, comprising:
a host configured to execute program instructions associated with an application, said host having a first IrDA interface; and
a biopsy driver assembly having a controller for executing program instructions and a user interface providing user input to said controller, said biopsy driver assembly having a second IrDA interface, said second IrDA interface being default disabled;
said controller of said biopsy driver assembly having sole control in enabling said second IrDA interface to in turn enable an infrared communications link between said first IrDA interface of said host and said second IrDA interface of said biopsy driver assembly.

2. The biopsy system of claim 1, wherein said application executing on said host provides password protected access to said biopsy driver assembly when said infrared communications link is enabled.

3. A biopsy system, comprising:
a host configured to execute program instructions associated with an application, said host having a host memory;
a biopsy driver assembly having a controller, firmware, a driver memory, and an event log established in said driver memory, said firmware having program instructions which when executed by said controller update said event log to record events related to usage of said biopsy driver assembly; and
an infrared communications link for facilitating communication between said host and said biopsy driver assembly,
said host executing program instructions from said application to retrieve said event log from said biopsy driver assembly over said infrared communications link.

4. The biopsy system of claim of claim 3, wherein said infrared communications link includes a first IrDA interface at said host and a second IrDA interface at said biopsy driver assembly, said biopsy driver assembly having a user interface, said second IrDA interface being as default disabled and is only enabled by a specific command entered at said user interface of said biopsy driver assembly.

5. The biopsy system of claim 3, wherein said event log is one of a plurality of event logs, each of said plurality of event logs storing data associated with a date and time of an occurrence of a respective biopsy event.

6. The biopsy system of claim 5, wherein said respective biopsy event includes an event associated with a tissue sample harvesting operation.

7. The biopsy system of claim 5, wherein said respective biopsy event includes an event associated with a piercing shot operation.

8. The biopsy system of claim 5, wherein each of said plurality of event logs stores an event count associated with said respective biopsy event.

9. The biopsy system of claim 5, said host executing program instructions from said application to analyze said plurality of event logs to determine a pattern of usage of said biopsy driver assembly.

10. The biopsy system of claim 3, said biopsy driver assembly having a parameter set stored in memory, said parameter set including at least one of a serial number of said biopsy driver assembly, a firmware version identification number of said biopsy driver assembly, a real time clock setting of said biopsy driver assembly, and motor positions of a plurality of motors of said biopsy driver assembly.

11. The biopsy system of claim 3, said biopsy driver assembly having a parameter set stored in memory, said parameter set including parameters associated with a plurality of motors in said biopsy driver assembly, said host executing program instructions from said application to selectively read at least one parameter in said parameter set associated with a respective motor of said plurality of motors.

12. The biopsy system of claim 3, said biopsy driver assembly having a parameter set stored in memory associated with a plurality of motors in said biopsy driver assembly, said host executing program instructions from said application to modify at least one parameter in said parameter set associated with a respective motor of said plurality of motors.

13. The biopsy system of claim 3, said host executing program instructions from said application to selectively control a plurality of functions of said biopsy driver assembly from said host during production assembly of said biopsy driver assembly, or service of said biopsy driver assembly, for testing said biopsy driver assembly.

14. The biopsy system of claim 13, wherein said plurality of functions include testing each of a plurality of motors and an associated plurality of drives in said biopsy driver assembly.

15. The biopsy system of claim 14, wherein said testing includes at least one of conducting driver operation sequences of said plurality of drives, measuring motor currents of said plurality of motors, and performing automatic motor adjustment and calibration of said plurality of motors by changing motor position parameters.

16. The biopsy system of claim 3, wherein said biopsy driver assembly has sole control in enabling said infrared communications link, and said application executing on said host facilitates password protected access to said biopsy driver assembly.

17. The biopsy system of claim 3, wherein said application is configured to provide data safety with checksum and data echo for verification of information retrieved from said biopsy driver assembly.

18. The biopsy system of claim 3, wherein said host memory has stored therein a respective descriptive file for each of a plurality of different types of said biopsy driver assembly, each type of biopsy driver assembly being identified by a unique driver identification number.

19. The biopsy system of claim 18, wherein each said descriptive file includes a listing of:
a number of parameters, parameter data types, read-only restrictions, and a description of parameters associated with a specific biopsy driver type of said plurality of different types of said biopsy driver assembly;
a number of event counters, a data type for each respective event counter, and a description of each respective event associated with said specific biopsy driver type;
a number of rows in each event log, a data type for each respective event log, and an event index table having descriptions of each respective event log associated with said specific biopsy driver type;
a password to be used for logging onto each type of biopsy driver assembly;
a proprietary binary format used to read and change data associated with said specific biopsy driver type; and
a checksum to check for data consistency before using said descriptive file associated with said specific biopsy driver type.

20. The biopsy system of claim 3, said biopsy driver assembly having a cannula drive configured to advance a cutter cannula in a biopsy probe assembly, said event log recording each time said cannula drive is actuated.

21. The biopsy system of claim 3, said biopsy driver assembly having a sample basket drive configured to move a sample basket in a biopsy probe assembly, said sample basket being used to receive a severed tissue sample, said event log recording each time said sample basket drive is actuated.

22. The biopsy system of claim 3, said biopsy driver assembly having a lift drive configured to move a sample collection tank in a biopsy probe assembly for the retrieval of a tissue sample from a sample basket of said biopsy probe assembly, said event log recording each time said lift drive is actuated.

23. The biopsy system of claim 3, said biopsy driver assembly having a mode select drive configured to select between a piercing shot mode and a tissue harvesting mode, said event log recording each time said piercing shot mode is selected and each time said tissue harvesting mode is selected.
